(19) United States
(12) Patent Application Publication
(10) Pub. No.: US 2009/0203628 A1
(43) Pub. Date: Aug. 13, 2009

(54) Composition, Method And Kit For Treating Skin Disorders And Improving Skin Condition

(76) Inventor: Jan Marini, San Jose, CA (US)

Correspondence Address: WILSON SONSINI GOODRICH & ROSATI, 650 PAGE MILL ROAD, PALO ALTO, CA, 94304-1050, US

(21) Appl. No.: 12/030,160

(22) Filed: Feb. 12, 2008

(51) Int. Cl. A61K 31/56; A61K 31/60; A61K 38/06; A61P 17/00

(52) U.S. Cl. 514/18; 514/171; 514/161; 514/2

(57) ABSTRACT

The present invention provides a composition, method and kit for treating skin disorders and improving skin condition. The composition, method and kit is particularly suited for treating acne vulgaris, seborrhea, rosacea, hirsutism, psoriasis, oily skin, dry skin, discoloration, post inflammatory hyper pigmentation, sun spots, and wrinkles.

### TECHNICAL FIELD

The invention relates to the field of skin disorder treatment and enhancement of skin condition. More particularly, the invention provides compositions, methods for manufacture of said compositions, methods of using said compositions, and a kit for treating skin disorders and enhancing skin condition.

### BACKGROUND

Many people suffer from skin disorders or want to improve the condition of their skin. Some skin disorders and conditions that can affect both youth and adults include acne vulgaris, seborrhea, hirsutism, psoriasis, oily skin, dry skin, discoloration, post inflammatory hyper pigmentation, sun spots, and wrinkles. These skin disorders and conditions can cause significant negative mental and physical health issues, as well as become a significant cost burden when seeking treatment. Because of the importance of treating these skin disorders and conditions, a large number of products have been developed in an attempt to achieve better treatment. One strategy presented here is to develop treatments utilizing combinations of skin conditioning agents.

### SUMMARY OF THE INVENTION

The present invention provides innovative compositions, methods and kits for treating skin disorders and enhancing skin condition. In one embodiment of the invention, the composition is a topical composition including a 5-alpha reductase inhibitor and a keratolytic agent. In another embodiment of the invention, the composition is a topical composition comprising a 5-alpha reductase inhibitor and a peptide for enhancing skin condition.

In some embodiments of the invention, the 5-alpha reductase inhibitor can be zinc-PCA, spironolactone, azelaic acid, pyridoxal phosphate, biotin and pantethine. In a preferred embodiment of the invention, the 5-alpha reductase inhibitor is spironolactone. In one aspect of the invention, the concentration of spironolactone is from about 0.1% to 10% by weight of the total composition.

In other embodiments of the invention, the keratolytic agent can be benzoyl peroxide, a retinoid, a beta-hydroxy acid, and an alpha-hydroxy acid. In a preferred embodiment of the invention, the beta-hydroxy acid is salicylic acid. In one aspect of the invention, the concentration of salicylic acid is from about 0.5% to 10% by weight of the total composition. In another embodiment of the invention, the alpha hydroxy acid is glycolic acid. In one aspect of the invention, the concentration of alpha-hydroxy acid is from about 0.1% to 20% by weight of the total composition.

In a preferred embodiment of the invention, the topical composition includes the 5-alpha reductase inhibitor spironolactone, preferably at a concentration from about 0.1% to 10% by weight of the total composition and the keratolytic agent salicylic acid, preferably at a concentration from about 0.5% to 10% by weight of the total composition.

In some embodiments, the composition comprising a 5-alpha reductase inhibitor and a keratolytic agent further comprises a peptide. In preferred embodiments of the invention, the peptide is acylated. In even more preferred embodiments of the invention, the acylated peptide is myristoyl tripeptide-3, optionally at a concentration from about 0.2% to 10% by weight of the total composition.

In a preferred embodiment of the invention, the 5-alpha reductase inhibitor is spironolactone, the keratolytic agent is salicylic acid, and the acylated peptide is myristoyl tripeptide-3. Optionally, the concentration of salicylic acid can be from about 0.1% to 10% by weight of the total composition, the concentration of the salicylic acid can be from about 0.5% to 10% by weight of the total composition, and the myristoyl tripeptide-3 can be from about 0.2% to 10% by weight of the total composition.

In another embodiment of the invention, the topical composition comprises a 5-alpha reductase inhibitor and a peptide, wherein the peptide is acylated. In preferred embodiments of the invention, the acylated peptide is myristoyl tripeptide-3.

In further embodiments of the invention, the composition comprising a 5-alpha reductase inhibitor and a keratolytic agent or a 5-alpha reductase inhibitor and a peptide further comprises a lightening agent, wherein the lightening agent is hexylresorcinol. In still further embodiments of the invention, said composition can further include a moisturizer, an antioxidant, an energy source, a natural extract, a solubilizer, a surfactant, a thickening agent, a buffer, and a preservative.

The inventor realized that solubilizing spironolactone has been a challenge and the traditional way of solubilizing spironolactone by using a concentrated alcohol is cosmetically undesirable as the high alcohol-content causes excessive drying of the skin.

According to the present invention, the compositions of the present invention preferably contain butylene glycol, which not only solubilizes spironolactone, but also circumvents the above described problems associated with using highly concentrated alcohol. The absence of an alcohol-based solubilizer in the composition avoids excessive drying of the skin and encourages enhancement of skin condition.

The topical compositions of the present invention can be formulated for use in the form of an aqueous solution, an emulsion, a gel, a lotion, a cream, an aerosol, a solid stick, a spray, or a patch. The formulation of the composition can be altered using methods known to those with ordinary skill in the art.

In another aspect of the invention, a method for treating skin disorders and enhancing skin condition is provided. The method to treat skin disorders and enhance skin conditions comprises applying a topical composition of the present invention to a skin area. In some embodiments of the invention, the method to treat skin disorders and enhance skin conditions comprises applying a topical composition that comprises a 5-alpha reductase inhibitor and a keratolytic agent. The 5-alpha reductase inhibitor can include zinc-PCA, spironolactone, azelaic acid, pyridoxal phosphate, biotin and pantethine. In preferred embodiments of the invention, the 5-alpha reductase inhibitor is spironolactone, preferably at a concentration from about 0.1% to 10% by weight of the total composition. In some embodiments of the invention, the keratolytic agent can include benzoyl peroxide, a retinoid, a beta-hydroxy acid, and an alpha-hydroxy acid. In preferred embodiments of the invention, the beta-hydroxy acid is salicylic acid, preferably at a concentration from about 0.5% to 10% by weight of the total composition. In other embodiments of the invention, the alpha-hydroxy acid is glycolic acid. In even more preferred embodiments of the invention, the method to treat skin conditions comprises applying a topical composition comprising a 5-alpha reductase inhibitor and a keratolytic agent, where the 5-alpha reductase inhibitor is spironolactone and the keratolytic agent is salicylic acid. Optionally, the concentration of spironolactone can be from about 0.1% to 10% by weight of the total composition and the concentration of salicylic acid can be from about 0.5% to 10% by weight of the total composition.

In preferred embodiments of the invention, the method to treat skin conditions includes applying a topical composition comprising a 5-alpha reductase inhibitor, a keratolytic agent, and a peptide, wherein said peptide can be an acylated peptide. In preferred embodiments of the invention, the acylated peptide is myristoyl tripeptide-3, preferably at a concentration from about 0.2% to 10% by weight of the total composition.

In even more preferred embodiments of the invention, the method to treat skin conditions includes applying a topical composition comprising a 5-alpha reductase inhibitor, a keratolytic agent, and a peptide, wherein the 5-alpha reductase inhibitor is spironolactone, the keratolytic agent is salicylic acid, and the peptide is an acylated peptide, wherein the acylated peptide is myristoyl tripeptide-3. Optionally, the concentration of salicylic acid can be about 0.1% to 10% by weight of the total composition, the concentration of salicylic acid can be about 0.5-10% by weight of the total composition and the concentration of myristoyl tripeptide-3 can be about 0.2% to 10% by weight of the total composition.

In further embodiments of the invention, the method to treat skin conditions including applying a topical composition comprising a 5-alpha reductase inhibitor and a keratolytic agent further comprises an anesthetic, wherein the anesthetic can be hexylresorcinol. In still further embodiments of the invention, the method to treat skin conditions including applying a topical composition comprising a 5-alpha reductase inhibitor and a keratolytic agent further can comprise a moisturizer, an antioxidant, an energy source, a natural extract, a solubilizer, a surfactant, a thickening agent, a buffer, and a preservative. In some embodiments of the invention, the solubilizer is butylene glycol.

In other embodiments of the invention, the method to treat skin conditions including applying a topical composition comprising a 5-alpha reductase inhibitor and a keratolytic agent comprises formulating the composition for use in the form of an aqueous solution, an emulsion, a gel, a lotion, a cream, an aerosol, a solid stick, a spray or a patch.

In some embodiments of the invention, the method to treat skin conditions including applying a topical composition comprising a 5-alpha reductase inhibitor and a keratolytic agent further comprises treating skin conditions like acne vulgaris, seborrhea, rosacea, hirsutism, psoriasis, oily skin, dry skin, discoloration, post inflammatory hyper pigmentation, sun spots, and wrinkles.

In other embodiments of the invention, the method to treat skin conditions including applying a topical composition comprising a 5-alpha reductase inhibitor and a keratolytic agent further comprises applying said topical composition to the head, face, neck, shoulder, arms, hands, back chest, stomach, buttocks, genitals, legs, and feet.

In other embodiments of the invention, the method to treat skin conditions comprises applying a topical composition that comprises a 5-alpha reductase inhibitor and a peptide. In some embodiments of the invention, the method to treat skin conditions comprises applying a 5-alpha reductase inhibitor and a peptide, wherein the 5 alpha reductase inhibitor is spironolactone and the peptide is myristoyl tripeptide-3.

In other embodiments of the invention, the method to treat skin conditions including applying a topical composition comprising a 5-alpha reductase inhibitor and a peptide comprises formulating the composition for use in the form of an aqueous solution, an emulsion, a gel, a lotion, a cream, an aerosol, a solid stick, a spray or a patch.

In still further embodiments of the invention, the method to treat skin conditions including applying a topical composition comprising a 5-alpha reductase inhibitor and a peptide further can comprise an anesthetic, a moisturizer, an antioxidant, an energy source, a natural extract, a solubilizer, a surfactant, a thickening agent, a buffer, and a preservative. In some embodiments of the invention, the solubilizer is butylene glycol.

In yet another aspect of the invention, a kit for treating skin disorders and enhancing skin condition is provided. The kit to treat skin disorders and enhance skin conditions comprises a container with a topical composition comprising a) i) a 5-alpha reductase inhibitor and a keratolytic agent or ii) a 5-alpha reductase inhibitor and a peptide and b) instructions for use.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

### Overview of the Invention

The present invention provides novel compositions, methods and kits for treating skin disorders and enhancing skin condition. The composition of the invention contains a 5-alpha reductase inhibitor (e.g. spironolactone) in combination with either a keratolytic agent (e.g. salicylic acid) or a peptide useful for proper skin maintenance. In some embodiments of the invention, the compositions, methods and kits can be used for treatment, prevention and/or the reduction of risk of various skin disorders, including but not limited to acne (e.g. acne vulgaris), oil control (e.g. oily skin), seborrhea, hirsutism, psoriasis, dry skin, discoloration (e.g., by post inflammatory hyper pigmentation from acne or other discolorations), sun spots, and wrinkles. In one aspect of the invention, the compositions of the present invention have been formulated to have a proper balance of efficacy, bioavailability, safety and comfort for long term or daily use.

When the term “about” is used in describing a value or an end-point of a range, the disclosure should be understood to include the specific value or end-point referred to.

The term “subject” means a mammal, preferably a human. The subject can be a human consumer or patient. More preferably, the subject is a human consumer.

### The Topical Composition

The present invention utilizes a combination of agents which are useful in the treatment, prevention and/or the reduction of risk of various skin disorders. In one embodiment of the invention, the topical composition comprises a 5-alpha reductase inhibitor (e.g. spironolactone) and a keratolytic agent (e.g. salicylic acid). In another embodiment of the invention, the topical composition comprises a 5-alpha reductase inhibitor and a peptide useful for proper skin maintenance.

5-alpha reductase inhibitors are a class of agents that block the function of the enzyme 5-alpha reductase. 5-alpha reductase acts to convert testosterone to dihydrotestosterone, a biologically active metabolite that participates in the development of male sex-characteristics. Hyperactive production of dihydrotestosterone is known to participate in the development of alopecia, seborrhea, hirsutism, and acne vulgaris. The use of 5-alpha reductase inhibitors for treatment of acne has been described in U.S. Pat. No. 6,174,892.

Members of the class of 5-alpha reductase inhibitors include, but are not limited to spironolactone, zinc-PCA, azelaic acid, pyridoxal phosphate, biotin, and pantethine. In one embodiment of the invention, a 5-alpha reductase inhibitor is selected from the group consisting of spironolactone, zinc-PCA, azelaic acid, pyridoxal phosphate, biotin, and pantethine. In other embodiments of the invention, the 5-alpha reductase inhibitor can be any agent that reduces that activity of 5-alpha reductase, preferably at an effective concentration.

Preferably, the 5-alpha reductase inhibitor is present at a concentration of from about 0.01 to about 20% by weight. More preferably, the 5-alpha reductase inhibitor is present in the compositions of the current invention at a concentration of from about 0.1% to 10% by weight, or even more preferably at a concentration of from about 1.0% to about 9.0%, 2.0% to about 8.0%, 3.0% to about 7.0%, or 4.0% to 6.0% by weight of the total composition. In a preferred embodiment of the invention, the 5-alpha reductase inhibitor is spironolactone. For example, in one embodiment of the invention, spironolactone is present in the compositions of the current invention at a concentration of from about 0.01 to about 20% by weight. More preferably, the spironolactone is present in the compositions of the current invention at a concentration of from about 0.1% to 10% by weight, or even more preferably at a concentration of from about 1.0% to about 9.0%, 2.0% to about 8.0%, 3.0% to about 7.0%, or 4.0% to 6.0% by weight of the total composition.

Previous topical compositions for treating skin disorders and skin conditions have not utilized non-alcohol agents for solubilization of spironolactone. The absence of an alcohol-based solubilizer encourages enhancement of skin condition and reduces that degree of detrimental side effects. The inventor realized that solubilizing spironolactone has been a challenge and the traditional way of solubilizing spironolactone by using a concentrated alcohol solution (such as one containing 50-80% alcohol) is cosmetically undesirable as the high alcohol-content causes excessive drying of the skin.

The compositions of the present invention preferably solubilize the 5-alpha reductase inhibitor (e.g. spironolactone) in butylene glycol, which not only solubilizes spironolactone, but also circumvents the above described problems associated with using highly concentrated alcohol.

In another embodiment of the invention, the 5-alpha reductase inhibitor is zinc-PCA. Zinc-PCA, available under the trade name ZINCIDONE, is the zinc salt of L-pyrrolidone carboxylic acid. Zinc-PCA inhibits 5-alpha reductase, helps regulate the activity of the sebaceous glands and reduces the level of skin sebum in vivo. Preferably, the zinc-PCA is present in the compositions of the current invention at a concentration of from about 0.01 to about 5% by weight. More preferably, the zinc-PCA is present in the compositions of the current invention at a concentration of from about 0.1% to 4% by weight, or even more preferably at a concentration of from about 0.5% to about 3.5%, 1.0% to about 3.0%, or 1.5% to 2.5% by weight of the total composition.

In another embodiment of the invention, the 5-alpha reductase inhibitor is azelaic acid. Azelaic acid is a well-tolerated agent that can be found naturally in wheat, rye and barley. Preferably, the azelaic acid is present in the compositions of the current invention at a concentration of from about 0.01 to about 30% by weight. More preferably, the azelaic acid is present in the compositions of the current invention at a concentration of from about 0.1% to 25% by weight, or even more preferably at a concentration of from about 1.0% to about 20%, 2.0% to about 15.0%, 3.0% to about 12.5%, 4.0% to about 10.0% or 5.0% to 7.5% by weight of the total composition.

In another embodiment, the 5-alpha reductase inhibitor is pyridoxal phosphate. Preferably, the pyridoxal phosphate is present in the compositions of the current invention at a concentration of from about 0.01 to about 30% by weight. More preferably, the pyridoxal phosphate is present in the compositions of the current invention at a concentration of from about 0.1% to 25% by weight, or even more preferably at a concentration of from about 1.0% to about 20%, 2.0% to about 15.0%, 3.0% to about 12.5%, 4.0% to about 10.0% or 5.0% to 7.5% by weight of the total composition.

In another embodiment, the 5-alpha reductase inhibitor is biotin. Preferably, the biotin is present in the compositions of the current invention at a concentration of from about 0.01 to about 30% by weight. More preferably, biotin is present in the compositions of the current invention at a concentration of from about 0.01% to 25% by weight, or even more preferably at a concentration of from about 0.05% to about 20%, 0.1% to about 15.0%, 0.5% to about 12.5%, 1% to about 10.0% or 2% to 7.5% by weight of the total composition.

In another embodiment, the 5-alpha reductase inhibitor is pantethine. Preferably, the pantethine is present in the compositions of the current invention at a concentration of from about 0.01 to about 30% by weight. More preferably, the pantethine is present in the compositions of the current invention at a concentration of from about 0.01% to 20% by weight, or even more preferably at a concentration of from about 0.05% to about 17.5%, 0.1% to about 15.0%, 0.2% to about 12.5%, 0.5% to about 1% or 1.5% to 7.5% by weight of the total composition.

The class of 5-alpha reductase inhibitors may be present alone or combination with other 5-alpha reductase inhibitors in the composition. The use of combinations of 5-alpha reductase inhibitors may be to increase overall efficacy of the composition or decrease the concentration required of each component, which may also decrease side effects associated with each agent. The 5-alpha reductase inhibitors may act additively or synergistically to achieve the desired effect. For example, pyridoxal phosphate can be used with zinc-PCA.

Previous treatments of skin conditions and skin disorders have not utilized combinations of a 5-alpha reductase inhibitor and a keratolytic agent, or of a 5-alpha reductase inhibitor and a peptide. The combination of a 5-alpha reductase inhibitor and a keratolytic agent or a 5-alpha reductase inhibitor and a peptide would better treat skin conditions then either component alone. The combination of a 5-alpha reductase inhibitor and a keratolytic agent would allow for effective treatment of acne and other skin conditions, while reducing the degree of detrimental side effects. The combination of a 5-alpha reductase inhibitor and a peptide would allow for treatment of a number of skin conditions while enhancing overall skin condition.

Keratolytic agents are a class of compounds that act as peeling agents to improve skin condition by inducing sloughing off of epithelium. Keratolytic agents have been used for treatment of a variety of skin conditions. These conditions include acne vulgaris, warts, corns, eczema, psoriasis, and seborrheic dermatitis. In follicles, keratolytic agents prevent clogging by slowing down shedding of cells. The use of keratolytic agents for treatment of acne has been described in U.S. Pat. No. 5,976,565.

Members of the class of keratolytic agents include, but are not limited to benzoyl peroxide, a retinoid, a beta-hydroxy acid, and an alpha-hydroxy acid. In other embodiments of the invention, the keratolytic agent can be any agent that acts as a peeling agent by inducing sloughing off of the epithelium, preferably at an effective concentration.

In one embodiment of the invention, the keratolytic agent is a beta-hydroxy acid. Preferably, the beta-hydroxy acid is present in the compositions of the current invention at a concentration of from about 0.1% to about 20% by weight. More preferably, the beta-hydroxy acid is present in the compositions of the current invention at a concentration of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition.

In a preferred embodiment of the invention, the beta-hydroxy acid is salicylic acid. For example, salicylic acid is present in the compositions of the current invention at a concentration of from about 0.1% to about 20% by weight. More preferably, the salicylic acid is present in the compositions of the current invention at a concentration of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition.

In another embodiment of the invention, the keratolytic agent is an alpha-hydroxy acid. Preferably, the alpha-hydroxy acid is present in the compositions of the current invention at a concentration of from about 0.1% to about 20% by weight. More preferably, the alpha-hydroxy acid is present in the compositions of the current invention at a concentration of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition. In a preferred embodiment of the invention, the alpha-hydroxy acid is glycolic acid. For example, glycolic acid may be present in the compositions of the current invention at a concentration of from about 0.1% to about 20% by weight. More preferably, the glycolic acid may be present in the compositions of the current invention at a concentration of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition.

In another embodiment of the invention, the keratolytic agent is a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. Examples of retinoids include, but are not limited to, retinol, retinol esters (e.g., C2-C22 alkyl esters of retinol, including retinyl palmitate, retinyl acetate, retinyl propionate), retinal, and/or retinoic acid (including all-trans retinoic acid and/or 13-cis-retinoic acid), and retinoids other than retinoic acid. Preferred retinoids are retinol, retinyl palmitate, retinyl acetate, retinyl propionate, retinal and combinations thereof. Preferably, the retinoid is present in the compositions of the current invention at a concentration of from about 0.1% to about 20% by weight. More preferably, the retinoid is present in the compositions of the current invention at a concentration of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition.

In another embodiment of the invention, the keratolytic agent comprises benzoyl peroxide. Benzoyl peroxide treats acne by not only facilitating keratolysis, but also working as an antibacterial. Preferably, the benzoyl peroxide is present in the compositions of the current invention at a concentration of from about 0.1% to about 20% by weight. More preferably, the benzoyl peroxide is present in the compositions of the current invention at a concentration of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition.

A peptide can enhance skin condition by, for example, reducing the effect of skin aging. The effects of skin aging are reduced by the encouragement of collagen production by the peptide, which reduces the presence of wrinkles, stretch marks, and dark circles. The use of a peptide to enhance skin condition has been described in U.S. Pat. Nos. 6,974,799 and 6,492,326. By peptide, the invention includes a peptide, a protein, a polypeptide (including an acylated peptide), a protein hydrolysate, a derivative and salt of a protein and a polypeptide, as well as combinations thereof.

A particularly preferred acylated peptide is comprised of (a) less than about ten amino acid residues, preferably less than about eight and (b) a C₁₂-C₂₂ acyl group, preferably C₁₄-C₁₆. In an even more preferable embodiment, the acylated peptide is myristoyl tripeptide-3, preferably at a concentration of from about 0.01% to 20% by weight, of from about 0.2% to 10% by weight, more preferably at a concentration of from about 1% to about 5% by weight of the total composition.

Tri-, tetra-, penta-, hexa- and heptapeptides as well as oligopeptides (including palmitoyl and myristoyl derivatives thereof) suitable for incorporation in compositions of the present invention are commercially-available from a number of suppliers and are described in the International Cosmetic Ingredient Dictionary and Handbook (10th Edition, 2003) published by the Cosmetic, Toiletry and Fragrance Association (“INCI Dictionary”).

One embodiment of the present invention is directed to a topical composition containing a 5-alpha reductase inhibitor, wherein the 5-alpha reductase inhibitor is spironolactone, preferably present at a concentration of from about 0.01 to about 20% by weight, or from about 0.1% to 10% by weight, or even more preferably at a concentration of from about 1.0% to 9.0%, 2.0% to about 8.0%, 3.0% to about 7.0%, or 4.0% to 6.0% by weight of the total composition, and a keratolytic agent, wherein the keratolytic agent is salicylic acid, preferably at a concentration of from about 0.1% to 20% by weight, of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition.

Another embodiment of the present invention is directed to a topical composition that contains a 5-alpha reductase inhibitor, wherein the 5-alpha reductase inhibitor is spironolactone, preferably present at a concentration of from about 0.01 to about 20% by weight, or from about 0.1% to 10% by weight, or even more preferably at a concentration of from about 1.0% to 9.0%, 2.0% to about 8.0%, 3.0% to about 7.0%, or 4.0% to 6.0% by weight of the total composition, and a peptide, wherein the peptide is myristoyl tripeptide-3, preferably at a concentration of from about 0.01% to 20% by weight, of from about 0.2% to 10% by weight, more preferably at a concentration of from about 1% to about 5% by weight of the total composition.

Yet another embodiment of the present invention is directed to a topical composition containing a 5-alpha reductase inhibitor, wherein the 5-alpha reductase inhibitor is spironolactone, preferably present at a concentration of from about 0.01 to about 20% by weight, of from about 0.01 to about 20% by weight, or from about 0.1% to 10% by weight, or even more preferably at a concentration of from about 1.0% to 9.0%, 2.0% to about 8.0%, 3.0% to about 7.0%, or 4.0% to 6.0% by weight of the total composition, and a keratolytic agent, wherein the keratolytic agent is salicylic acid, preferably at a concentration of from about 0.1% to 20% by weight, of from about 0.5% to 10% by weight, or even more preferably at a concentration of from about 0.6% to about 9.0%, 0.7% to about 7.5%, 0.8% to about 5.0%, 0.9% to about 2.5% or 1.0% to 2.0% by weight of the total composition, and the peptide is myristoyl tripeptide-3, preferably at a concentration of from about 0.01% to 20% by weight, of from about 0.2% to 10% by weight, more preferably at a concentration of from about 1% to about 5% by weight of the total composition.

In some embodiments of the invention, the topical composition further comprises a lightening agent. A particularly preferred lightening agent is hexylresorcinol. The use of resorcinol derivatives for skin lightening has been described in US Patent Application 2005/0271608 and 2003/0190298. In some embodiments of the invention, the hexylresorcinol is preferably at a concentration of from about 0.01% to 10% by weight, of from about 0.2% to 5% by weight, more preferably at a concentration of from about 0.5% to about 3% by weight of the total composition.

The compositions of the present invention may also include other ingredients which may be useful for improving the condition of skin (e.g. moisturizers) or improving the overall pleasure of using the composition (e.g. perfumes). Thus, in various embodiments of the invention, the topical composition may further comprise a moisturizer, an antioxidant, an anti-inflammatory agent, an energy source, and a natural extract. A person of skill in the field would know and understand the various additional ingredients that may be used in a topical composition as well as the concentrations of each ingredient that may be used.

A moisturizer suitable for use in the composition of the present invention is well-known to those of ordinary skill in the art and include, but is not limited to: butylene glycol, propylene glycol, and caprylic/capric triglyceride. The concentration of the moisturizer can be in the amount from 1% to 90% of the total weight of the composition.

An antioxidant and/or anti-inflammatory agent suitable for use in the compositions of the present invention is well-known to those of ordinary skill in the art and include, but is not limited to: dipotassium glycyrrhetinate; allantoin; a flavonoid, including green tea extract; an enzyme, including superoxide dismutase, catalase and glutathione peroxidase; a vitamin and a vitamin derivative, including ascorbic acid, tocopherol, and a derivative thereof. The antioxidant and anti-inflammatory agent suitable for use in the compositions of the present invention may be in amount from about 0.01% to about 10%, more preferably from about 0.05% to about 5% by weight of the composition.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox™), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, lmethionine, proline, superoxide dismutase, silymarin, tea extracts, grape skin/seed extracts, melanin, and rosemary extracts may be used. Preferred anti-oxidants/radical scavengers are selected from tocopherol sorbate and other esters of tocopherol, more preferably tocopherol sorbate.

An energy source suitable for use in the composition of the present invention includes, but is not limited to: creatine, phosphocreatine, creatine ethyl ester, ATP, NTP. The energy source suitable for use in the compositions of the present invention may be in amount from about 0.01% to about 10%, more preferably from about 0.05% to about 5% by weight of the composition.

A natural extract suitable for use in the composition of the present invention include, but is not limited to: those mentioned previously, cucumis sativus (cucumber) fruit extract, arnica montana flower extract, anacyclus pyrethrum root extract (Spanish chamomile or Mount Atlas daisy), dipotassium glycyrrhizate (licorice extract), japanese green tea extract, omega-3 ceramide (linseed oil, palm oil aminopropanediol esters), linoleic acid and linolenic acid, and natural fragrances. The natural extract suitable for use in the compositions of the present invention may be in amount from about 0.01% to about 50%, more preferably from about 0.05% to about 15% by weight of the composition.

Additional components are used for solubilization of all components in a form usable for topical application and in a form that is stable over the lifetime of the product. Said components comprise a solubilizer, a surfactant, a thickening agent, a buffer, and a preservative.

In a preferred embodiment, at least one 5-alpha reductase inhibitor is spironolactone, preferably present at a concentration of from about 0.1% to 10% by weight of the total composition. The inventor realized that solubilizing spironolactone has been a challenge and the traditional way of solubilizing spironolactone by using a concentrated alcohol solution (such as one containing 50-80% alcohol) is cosmetically undesirable as the high alcohol-content causes excessive drying of the skin.

According to the present invention, the composition preferably contains butylene glycol, which not only solubilizes spironolactone, but also circumvents the above described problems associated with using highly concentrated alcohol. Another solubilizer that can be used includes, but is not limited to: butylene glycol, water, and propylene glycol. The solubilzer suitable for use in the compositions of the present invention may be in amount from about 1% to about 99%, more preferably from about 5% to about 90% by weight of the composition.

A surfactant suitable for use in the composition of the present invention includes, but is not limited to: steareth-100, steareth-2, polysorbate 20, and hydroxypropyl starch phosphate. The surfactant suitable for use in the compositions of the present invention may be in amount from about 0.01% to about 10%, more preferably from about 0.05% to about 5% by weight of the composition. The surfactant may also function as an emulsifier to stabilize the composition.

The compositions of the present invention may contain one or more of a thickening agent. Examples of the thickening agent include, but are not limited to, a carboxylic acid polymer, a cross-linked polyacrylate polymer, a polyacrylamide polymer, a polysaccharide and a naturally derived thickening agent.

Carboxylic acid polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the CARBOPOL™ 900 series from B.F. Goodrich (e.g., CARBOPOL™ 954). In addition, other suitable carboxylic acid polymeric agents include copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as CARBOPOL™1342, CARBOPOL™1382, PEMULEN TR-1, and PEMULEN TR-2, from B.F. Goodrich. In other words, examples of carboxylic acid polymer thickeners useful herein are those selected from carbomers, acrylates/C10-C30 alkyl acrylate crosspolymers, and mixtures thereof.

Crosslinked polyacrylate polymers useful as thickeners or gelling agents include both cationic and nonionic polymers, with the cationics being generally preferred. Examples of useful crosslinked nonionic polyacrylate polymers and crosslinked cationic polyacrylate polymers are those described in U.S. Pat. No. 5,100,660, to Hawe et al, issued Mar. 31, 1992; U.S. Pat. No. 4,849,484, to Heard, issued Jul. 18, 1989; U.S. Pat. No. 4,835,206, to Farrar et al, issued May 30, 1989; U.S. Pat. No. 4,628,078 to Glover et al issued Dec. 9, 1986; U.S. Pat. No. 4,599,379 to Flesher et al issued Jul. 8, 1986; and EP 228,868, to Farrar et al, published Jul. 15, 1987.

Polyacrylamide polymers, especially nonionic polyacrylamide polymers, include substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

A polysaccharide can be used as a thickening agent, which contains a backbone of repeating sugar (i.e., carbohydrate) units. Nonlimiting examples of a polysaccharide thickening agents include mannan, cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (preferably hydroxyethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol.®. CS Plus from Aqualon Corporation (Wilmington, Del.).

Other thickening and gelling agents useful herein include materials which are primarily derived from natural sources. Nonlimiting examples of these thickening agents include glycerol, acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

In some embodiments, compositions of the present invention include a thickening agent selected from a carboxylic acid polymer, a crosslinked polyacrylate polymer, a polyacrylamide polymer, a polysaccharide, a natural thickening agent, and a mixture thereof. The thickening agent suitable for use in the compositions of the present invention may be in amount from about 0.01% to about 20%, more preferably from about 1% to about 10% by weight of the composition.

In some embodiments of the invention, the topical composition further comprises a preservative and a buffer. A preservative suitable for use in the composition includes methylparaben, propylparaben, and diazolidinyl urea. The amount of preservative in the composition is preferably at a concentration of from about 0.01% to 10% by weight, more preferably at a concentration of from about 0.5% to about 3% by weight of the total composition. A buffer suitable for use in the composition includes sodium acetate, phosphate buffer, HEPES buffer, and TRIS buffer. The amount of preservative in the composition is preferably at a concentration of from about 0.01% to 10% by weight, more preferably at a concentration of from about 0.5% to about 3% by weight of the total composition.

The topical composition comprises adding ingredients in effective concentrations that have a proper balance of efficacy, bioavailability, safety and comfort for long term or daily use. In other embodiments of the invention, the ingredients are added in effective concentrations to form a cosmetic, cosmeceutical, therapeutic, or pharmaceutical composition. In yet other embodiments of the invention, the solubilizers, surfactants, and thickening agents are added in amounts for use of the topical composition in the form of an aqueous solution, an emulsion, a gel, a lotion, a cream, an aerosol, a solid stick, a spray or a patch.

The topical composition according to the present invention may be applied to the skin in any number of forms or vehicles known to those of skill in the art including as: (i) aqueous, aqueous-alcoholic or oily solutions, optionally gelled; (ii) an emulsion obtained by dispersing an aqueous phase in an oil or silicone phase, or vice versa (i.e., water-in-oil, oil-in-water, water-in-silicone, silicone-in-water); (iii) a triple emulsion, (i.e., water-in-oil-in-water or oil-in-water-in-oil); or (iv) a vesicular dispersion. The viscosity of the final formulation may be adjusted within the level of ordinary skill in the art to form a cream, lotion, gel, serum or spray.

Optionally, the composition is formulated for topical application, which can be in form of an aqueous solution, emulsion, gel, lotion, cream, aerosol, ointment, salve, patch, solid stick, or spray. As used herein, a “lotion” refers to a liquid, usually an aqueous medicinal preparation containing one or more insoluble substances and applied externally for skin disorders; “cream” refers to an emulsified medicinal or cosmetic preparation; a semisolid emulsion of either the oil-in-water or the water-in-oil type, ordinarily intended for topical use; “gel” refers to a colloid in a more solid form than a solution; a jelly-like material formed by the coagulation of a colloidal liquid; many gels have a fibrous matrix and fluid filled interstices: gels are viscoelastic rather than simply viscous and can resist some mechanical stress without deformation; and “ointment” refers to a salve or unguent for application to the skin, specifically a semisolid medicinal preparation usually having a base of fatty or greasy material; an ointment has an oil base whereas a cream is water-soluble. The composition may be used as a cosmetic, cosmeceutical, therapeutic, or pharmaceutical composition.

### Method of Use

Compositions of this invention are in the form of products to be applied to the skin or epithelium of subjects or patients, i.e. humans or other mammals in need of the particular treatment. The method of using the product comprises applying an effective amount of the topical composition to a skin area for the treatment, prevention and/or the reduction of risk of various skin disorders, including but not limited to acne (e.g. acne vulgaris), oil control (e.g. oily skin), seborrhea, hirsutism, psoriasis, dry skin, discoloration (e.g. by post-inflammatory hyper pigmentation from acne or other discolorations), sun spots, and wrinkles.

For example, the compositions may be applied in a thin layer on the skin covering the area to be treated. It is further contemplated that the compositions of the present invention may be integrated directly into packaged bandages for convenient application. The bandages of the invention come with the compositions of the invention pre-applied to allow for easy administration to a subject.

In one embodiments of the invention, the method of using the product comprises applying a first topical composition that comprises a 5-alpha reductase inhibitor and a keratolytic agent. In another embodiment of the invention, the method of using the product comprises applying a second topical composition that comprises a 5-alpha reductase inhibitor and a peptide. In yet another embodiment of the invention, the method of using the product comprises applying a third topical composition that comprises a 5-alpha reductase inhibitor, a keratolytic agent, and a peptide.

In other embodiments of the invention, the method of using the product comprises applying said first, second or third topical composition that further comprises a lightening agent, a moisturizer, an antioxidant, an energy source, a natural extract, a solubilizer, a surfactant, a thickening agent, a buffer, and a preservative.

Compositions according to the present invention may be applied to the skin in any number of forms or vehicles known to those of skill in the art including: (i) aqueous, aqueous-alcoholic or oily solutions, optionally gelled; (ii) emulsions obtained by dispersing an aqueous phase in an oil or silicone phase, or vice versa (i.e., water-in-oil, oil-in-water, water-in-silicone, silicone-in-water); (iii) triple emulsions (i.e., water-in-oil-in-water or oil-in-water-in-oil); or (iv) vesicular dispersions. The viscosity of the final formulation may be modulated by methods known in the art to form a cream, lotion, gel, serum or spray. Any form of applicator known in the art is also included in the methods of administering the compositions of the invention.

In some embodiments of the invention, the skin areas include, for example, the head, face, neck, shoulders, arms, hands, back, chest, stomach, buttocks, genitals, legs, and feet. In other embodiments of the invention, the method of using the product comprises applying an amount of the composition to an affected skin area. In some embodiments of the invention, the affected skin areas comprise areas of skin affected by acne vulgaris, seborrhea, hirsutism, psoriasis, oily skin, sun spots, and wrinkles.

Topical application may be more than about once, twice, three times, four times, five times, six times or seven times per week, or more than once, twice, three times, four times, five times, six times or seven times per day. Frequency of application may be less than about twice, three times, four times, five times, six times or seven times per week, or less than about once, twice, three times, four times, five times, six times or seven times per day. Some embodiments of the invention provide a method for cosmetic treatment of the skin of an individual by topical administration of an effective amount of the composition of the invention. In some embodiments, the composition is administered an average of about once per day; in some embodiments, the composition is administered an average of about once or twice per day; in some embodiments, the composition is administered an average of about once to three times per day; in some embodiments, the composition is administered an average of more than about three times per day. In one embodiment, the composition is administered an average of about twice per day, typically in the morning upon rising and in the evening before retiring. The composition may be applied and left on the skin. Alternatively, the composition may be applied and removed, for example, by washing the skin. The composition may also be applied by spot application, for example, directly to areas where necessary, or may be applied generally, to cover an entire skin region.

### Kits

In still another aspect, the present invention provides kits for the cosmetic treatment of skin or to produce a desired cosmetic result. These kits comprise the compositions described herein, in a container or containers which are held in suitable packaging. In some embodiments the kits further contain instructions teaching the use of the kit according to the various methods and approaches described herein. Such kits may also include information, such as scientific literature references, package insert materials, cosmetic trial results, and/or summaries of these and the like, which indicate or establish the activities and/or advantages of the composition. Such information may be based on the results of various studies, for example, studies using experimental animals involving in vivo models and studies based on human cosmetic or clinical trials. Kits described herein can be provided, marketed and/or promoted to health care providers (e.g., dermatologists and other physicians), skin care appearance care providers, including cosmetologists, hair stylists, and the like. Kits for cosmetic use may also be provided, marketed and/or promoted directly to consumers. Kits may be marketed in spas and retail outlets.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that methods and structures within the scope of these claims and their equivalents be covered thereby.

### EXAMPLES

### Example 1

### Treatment of Acne using Composition of the Present Invention

A subject having a diagnosed case of acne vulgaris is treated using the following composition (all amounts are percent by weight): 5% spironolactone; 0.5% salicylic acid, 15% butylene glycol, 5% antioxidants (dipotassium glycyrrhetinate and flavonoid mixture), 5% creatine, 5% natural extracts (including cucumber fruit extract, licorice extract and linoleic acid), and water to 100%. The subject is treated twice daily, by topical application, once in the morning and once in the evening. The subject is monitored daily for the reduction in size of acne blemishes as well as reduction in number and severity. The subject is monitored for six months.

### Example 2

### Treatment of Oily Skin using Composition of the Present Invention

A subject suffering from oily skin is treated using the following composition (all amounts are percent by weight): 3% spironolactone; 1% salicylic acid, 15% butylene glycol, 5% antioxidants (dipotassium glycyrrhetinate and flavonoid mixture), 5% creatine, 5% natural extracts (including cucumber fruit extract, licorice extract and linoleic acid), and water to 100%. The subject is treated twice daily, by topical application, once in the morning and once in the evening. The subject is monitored daily for the reduction in size of acne blemishes as well as reduction in number and severity. The subject is monitored for six months.

What is claimed is:

1. A topical composition comprising a 5-alpha reductase inhibitor and a keratolytic agent.

2. The topical composition of claim 1, wherein the 5-alpha reductase inhibitor is selected from the group consisting of zinc-PCA, spironolactone, azelaic acid, pyridoxal phosphate, biotin, and pantethine.

3. The topical composition of claim 1, wherein the 5-alpha reductase inhibitor is spironolactone.

4. The topical composition of claim 3, wherein the concentration of spironolactone is about 0.1% to 10% by weight of the total composition.

5. The topical composition of claim 1, wherein the keratolytic agent is selected from the group consisting of benzoyl peroxide, a retinoid, a beta-hydroxy acid, and an alpha-hydroxy acid.

6. The topical composition of claim 5, wherein the beta-hydroxy acid is salicylic acid.

7. The topical composition of claim 6, wherein the concentration of salicylic acid is about 0.5% to 10% by weight of the total composition.

8. The topical composition of claim 7, wherein the alpha-hydroxy acid is glycolic acid.

9. The topical composition of claim 1, wherein the 5-alpha reductase inhibitor is spironolactone and the keratolytic agent is salicylic acid.

10. The topical composition of claim 9, wherein the concentration of spironolactone is about 0.1% to 10% by weight of the total composition and the concentration of salicylic acid is about 0.5% to 10% by weight of the total composition.

11. The topical composition of claim 1 further comprising a peptide.

12. The topical composition of claim 11 wherein the peptide is acylated.

13. The topical composition of claim 12, wherein the acylated peptide is myristoyl tripeptide-3.

14. The topical composition of claim 13, wherein the concentration of myristoyl tripeptide-3 is about 0.2% to 10% by weight of the total composition.

15. The topical composition of claim 12, wherein the 5-alpha reductase inhibitor is spironolactone, the keratolytic is salicylic acid, and the acylated peptide is myristoyl tripeptide-3.

16. The topical composition of claim 15, wherein the concentration of spironolactone is about 0.1% to 10% by weight of the total composition, the concentration of salicylic acid is about 0.5% to 10% by weight of the total composition and the concentration of myristoyl tripeptide-3 is about 0.2% to 10% by weight of the total composition.

17. The topical composition of claim 1 further comprising hexylresorcinol.

18. The topical composition of claim 1 further comprising a component selected from the group of a moisturizer, an antioxidant, an energy source, a natural extract, a solubilizer, a surfactant, a thickening agent, a buffer, and a preservative.

19. The topical composition of claim 18, wherein the solubilizer is butylene glycol.

20. The topical composition of claim 18, wherein the composition is formulated for use in the form of an aqueous solution, an emulsion, a gel, a lotion, a cream, an aerosol, a solid stick, a spray or a patch.

21. A topical composition comprising 5-alpha reductase inhibitor and a peptide.

22. The topical composition of claim 21, wherein the 5-alpha reductase inhibitor is spironolactone and the peptide is myristoyl tripeptide-3.

23. The topical composition of claim 21 further comprising a component selected from the group of a lightening agent, a moisturizer, an antioxidant, an energy source, a natural extract, a solubilizer, a surfactant, a thickening agent, a buffer, and a preservative.

24. The topical composition of claim 23, wherein the solubilizer is butylene glycol.

25. The topical composition of claim 24, wherein the composition is formulated for use in the form of an aqueous solution, an emulsion, a gel, a lotion, a cream, an aerosol, a solid stick, a spray or a patch.

26. A method to treat a subject having a skin condition comprising applying to the skin of the subject a topical composition comprising a 5-alpha reductase inhibitor and a keratolytic agent.

27. The method of claim 26, wherein the 5-alpha reductase inhibitor is selected from the group consisting of zinc-PCA, spironolactone, azelaic acid, pyridoxal phosphate, biotin, and pantethine.

28. The method of claim 26, wherein the 5-alpha reductase inhibitor is spironolactone.

29. The method of claim 28, wherein the concentration of spironolactone is about 0.1% to 10% by weight of the total composition.

30. The method of claim 26, wherein the keratolytic agent is selected from the group consisting of benzoyl peroxide, a retinoid, a beta-hydroxy acid, and an alpha-hydroxy acid.

31. The method of claim 30, wherein the beta-hydroxy acid is salicylic acid.

32. The method of claim 31, wherein the concentration of salicylic acid is about 0.5% to 10% by weight of the total composition.

33. The method of claim 30, wherein the alpha-hydroxy acid is glycolic acid.

34. The method of claim 26, wherein the 5-alpha reductase inhibitor is spironolactone and the keratolytic agent is salicylic acid.

35. The method of claim 34, wherein the concentration of spironolactone is about 0.1% to 10% by weight of the total composition and the concentration of salicylic acid is about 0.5% to 10% by weight of the total composition.

36. The method of claim 26, comprising applying said topical composition further comprising a peptide.

37. The method of claim 36, wherein the peptide is acylated.

38. The method of claim 37, wherein the acylated peptide is myristoyl tripeptide-3.

39. The method of claim 38, wherein the concentration of myristoyl tripeptide-3 is about 0.2% to 10% by weight of the total composition.

40. The method of claim 37, wherein the 5-alpha reductase inhibitor is spironolactone, the keratolytic is salicylic acid, and the acylated peptide is myristoyl tripeptide-3.

41. The method of claim 40, wherein the concentration of spironolactone is about 0.1% to 10% by weight of the total composition, the concentration of salicylic acid is about 0.5-10% by weight of the total composition and the concentration of myristoyl tripeptide-3 is about 0.2% to 10% by weight of the total composition.

42. The method of claim 26, comprising applying said topical composition further comprising hexylresorcinol.

43. The method of claim 26, wherein the composition further comprises one or more of the following: a moisturizer, an antioxidant, an energy source, a natural extract, a solubilizer, a surfactant, a thickening agent, a buffer, or a preservative.

44. The method of claim 43, wherein said solubilizer is butylene glycol.

45. The method of claim 26, comprising applying said topical composition, wherein the composition is formulated for use in the form of an aqueous solution, an emulsion, a gel, a lotion, a cream, an aerosol, a solid stick, a spray or a patch.

46. The method of claim 26, wherein the skin condition is selected from the group consisting of acne vulgaris, seborrhea, rosacea, hirsutism, psoriasis, oily skin, dry skin, discoloration, sun spots, and wrinkles.

47. A method to treat a subject having a skin condition comprising applying to the skin of the subject a topical composition comprising a 5-alpha reductase inhibitor and a peptide.

48. The method of claim 47, wherein the 5-alpha reductase inhibitor is spironolactone and the peptide is myristoyl tripeptide-3.

49. The method of claim 47, wherein the composition further comprises a lightening agent, a moisturizer, an antioxidant, an energy source, a natural extract, a solubilizer, a surfactant, a thickening agent, a buffer, or a preservative.

50. The method of claim 47, wherein the solubilizer is butylene glycol.

51. The method of claim 47, comprising applying said topical composition, wherein the composition is formulated for use in the form of an aqueous solution, an emulsion, a gel, a lotion, a cream, an aerosol, a solid stick, a spray or a patch.

52. The method of claim 47, wherein the skin condition is selected from the group consisting of acne vulgaris, seborrhea, rosacea, hirsutism, psoriasis, oily skin, dry skin, discoloration, sun spots, and wrinkles.

53. A kit for treating a skin condition comprising a container, a 5-alpha reductase, a keratolytic agent and instructions.

54. A kit for treating a skin condition comprising a container, a 5-alpha reductase, a peptide and instructions.
